(19) United States
(12) Patent Application Publication
(10) Pub. No.: US 2019/0336486 A1
(43) Pub. Date: Nov. 7, 2019

(54) METHODS FOR TREATING CANCER AND METABOLIC SYNDROME

(71) Applicant: GLAX LLC, Wilmington, DE (US)

(72) Inventors: Rakesh K Srivastava, New Orleans, LA (US); Sharmila Shankar, New Orleans, LA (US); Sushant Kumar Shrivastava, Varanasi (IN); Pavan Srivastava, Varanasi (IN); Piyoosh Sharma, Varanasi (IN); Prabhash Nath Tripathi, Varanasi (IN)

(73) Assignee: GLAX LLC, Wilmington, DE (US)

(21) Appl. No.: 16/399,099

(22) Filed: Apr. 30, 2019

Related U.S. Application Data
(60) Provisional application No. 62/666,855, filed on May 4, 2018.

(51) Int. Cl. A61K 31/426

(52) U.S. Cl. CPC A61K 31/426

(57) ABSTRACT

This invention is directed to methods for treating cancer and metabolic syndrome.

This application claims priority from U.S. Provisional Application No. 62/666,855 filed on May 4, 2018.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to methods for treating cancer and metabolic syndrome.

### BACKGROUND OF THE INVENTION

Metabolic syndrome (MetS) comprises a cluster of risk factors that includes central obesity, dyslipidemia, impaired glucose homeostasis and hypertension. Obesity is strongly associated with the development of insulin resistance, which in turn plays a key role in the pathogenesis of obesity-associated cardiometabolic complications, including metabolic syndrome components, type 2 diabetes, and cardiovascular disease. Metabolic disorders and obesity have been linked to cancer. Brain tumor is abnormal growth of cells in the brain. The World Health Organization report estimated 7.6 million people died from cancer accounting for the 13% of all deaths worldwide due to this disease.

### SUMMARY OF THE INVENTION

The present invention provides a method of treating a subject afflicted with a disease, comprising administering to a subject in need thereof a therapeutically effective amount of a 3-adrenomimetic agent, wherein the disease is not overactive bladder. Non-limiting examples of such diseases comprise metabolic syndrome, obesity, diabetes, and cancer.

The present invention further provides a method of preventing the progression of cancer in a subject, comprising administering to a subject in need thereof a therapeutically effective amount of a 3-adrenomimetic agent. Non-limiting examples of a 3-adromimetic agent comprises mirabegron.

In embodiments, the agent induces apoptosis of a cancer cell, inhibits proliferation of a cancer cell, inhibits migration of a cancer cell, or a combination thereof.

In embodiments, the cancer cell comprises a cancer stem cell.

In embodiments, the cancer is a solid tumor. In embodiments, the cancer is a liquid tumor.

In embodiments, the cancer comprises a brain cancer (such as glioma or glioblastoma), breast cancer, prostate cancer, lung cancer, leukemia, lymphoma, ovarian cancer, neuroendocrine cancer, colon cancer, colorectal cancer, bladder cancer, endometrial cancer, neuroblastoma, kidney cancer, multiple myeloma, pancreatic cancer, thyroid cancer, liver cancer, gastric cancer, or melanoma.

In embodiments, he agent contacts and/or interacts with the SMO receptor. Non-limiting examples of such contacts and/or interactions comprise a hydrogen bond, a hydrophobic interaction, an ionic interaction, water bridge formation, or a combination thereof.

In embodiments, the agent contacts and/or interacts with the SMO receptor at Tyr394, Lys395, Asp473, Glu481, Glh518 or any combination thereof. For example, the interaction comprises a hydrogen bond.

In embodiments, the agent contacts and/or interacts with the SMO receptor at Tyr207, Ile215, Leu221, Trp281, Ile389, Phe391, Trp394, Arg400, His470, Phe484, Leu522, or any combination thereof. For example, the interaction comprises a hydrophobic interaction.

In embodiments, the interactions have been observed through in silico molecular docking and dyanmics simulations studies using Schrödinger Suite 10.5.014 MM Share Version 3.3.014 Release 2016-1.

In embodiments, the agent contacts and/or interacts with the SMO receptor at Asp473.

In embodiments, the agent contacts and/or interacts with the SMO receptor at Asp384, Val517, Asn 521, or any combination thereof.

The invention is also directed towards a method of treating a subject afflicted with a disease, comprising administering to a subject in need thereof a therapeutically effective amount of an agent that contacts and/or interacts with the SMO receptor, wherein the disease is not overactive bladder. Non-limiting examples of such diseases comprise metabolic syndrome, obesity, diabetes, and cancer. Non-limiting examples of such contacts and/or interactions comprise a hydrogen bond, a hydrophobic interaction, an ionic interaction, water bridge formation, or a combination thereof.

In embodiments, the agent contacts and/or interacts with the SMO receptor at Tyr394, Lys395, Asp473, Glu481, Glh518, Tyr207, Ile215, Leu221, Trp281, Ile389, Phe391, Trp394, Arg400, His470, Phe484, Leu522, Asp384, Val517, Asn 521 or any combination thereof.

In embodiments, treating comprises preventing the progression of cancer in the subject.

In embodiments, the agent induces apoptosis of a cancer cell, inhibits proliferation of a cancer cell, inhibits migration of a cancer cell, or a combination thereof.

In embodiments, the agent comprises a 3-adrenomimetic agent, such as mirabegron.

In embodiments, the cancer is a solid tumor. In embodiments, the cancer is a liquid tumor.

In embodiments, the cancer comprises a brain cancer (such as glioma or glioblastoma), breast cancer, prostate cancer, lung cancer, leukemia, lymphoma, ovarian cancer, neuroendocrine cancer, colon cancer, colorectal cancer, bladder cancer, endometrial cancer, neuroblastoma, kidney cancer, multiple myeloma, pancreatic cancer, thyroid cancer, liver cancer, gastric cancer, or melanoma.

Aspects of the invention are also directed towards methods of treating a neurological disorder comprising administering to a subject afflicted with a neurological disorder a therapeutically effective amount of a compound described herein.

Other objects and advantages of this invention will become readily apparent from the ensuing description.

### BRIEF DESCRIPTION OF THE FIGURES

FIG. 1 shows the binding pose predicted by Glide-XP of SMO receptor (A) and Standard LY2940680

FIG. 2 shows 3-D binding pose predicted by Glide-XP of SMO receptor (A) and Mirabegron

FIG. 3 shows 2-D binding pose predicted by Glide-XP of SMO receptor (A) and Mirabegron

FIG. 4 shows RMSD fluctuations of protein backbone (green) and Mirabegron (Red) for 10 ns simulation run on 4JKV.

FIG. 5 shows Protein-Ligand Contacts of Mirabegron for 10 ns simulation run on 4JKV.

FIG. 6 shows Ligand-Protein Contacts of Mirabegron for 10 ns simulation run on 4JKV.

FIG. 7 shows Mirabegron inhibits cell proliferation of glioblastoma cell lines and brain CSCs.

FIG. 8 shows Mirabegron inhibits colony formation by glioblastoma cells and brain CSCs.

FIG. 9 shows Mirabegron inhibits cell proliferation of lung cancer cells (A549), but has no effect on human normal lung epithelial cells (Beas 2B).

FIG. 10 shows Mirabegron inhibits colony formation by lung cancer cells.

FIG. 11 shows Mirabegron inhibits cell proliferation of colorectal CSCs.

FIG. 12 shows Mirabegron inhibits cell proliferation of breast cancer cells.

FIG. 13 shows Mirabegron inhibits cell proliferation of prostate cancer cells.

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are directed towards methods of treating a subject afflicted with a disease, such as cancer, metabolic disorders, diabetes, and/or obesity, comprising administering to a subject in need thereof a therapeutically effective amount of a 3-adrenomimetic agent or an agent that contacts and/or interacts with the SMO receptor. The terms “treat,” “treating” or “treatment”, can include, but are not limited to, prophylactic and/or therapeutic treatments.

In embodiments, the diseases comprises cancer. As used herein, the terms “tumor” and “cancer” can be used interchangeably, and generally refer to a physiological condition characterized by the abnormal and/or unregulated growth, proliferation or multiplication of cells.

In embodiments, the didese comprises a metabolic disorder. As used herein, a “metabolic disorder” can refer to any pathological condition resulting from an alteration in a subject's metabolism. Such disorders include those resulting from an alteration in glucose homeostasis and/or insulin dysfunction. Metabolic disorders, include but are not limited to, metabolic syndrome, elevated blood glucose levels, insulin resistance, glucose intolerance, type 2 diabetes, type 1 diabetes, pre-diabetes, non-alcoholic fatty liver disease, nonalcoholic steatohepatitis, and obesity.

The terms “treat,” “treating” or “treatment” can refer to the lessening of severity of a disease, delay in onset of a disease, slowing the progression of a disease, shortening of duration of a disease, relieving a condition caused by a disease, or stopping symptoms which result from a disease.

A “therapeutically effective amount” of a 3-adrenomimetic agent or an agent that contacts and/or interacts with the SMO receptor can refer to an amount sufficient to provide a benefit in the treatment of a disease, to delay or minimize symptoms associated with a disease, or to cure or ameliorate a disease. In particular, a therapeutically effective amount means an amount of a 3-adrenomimetic agent or an agent that contacts and/or interacts with the SMO receptor sufficient to provide a therapeutic benefit in vivo. The term preferably encompasses a non-toxic amount of a 3-adrenomimemtic agent that improves overall therapy, reduces or avoids symptoms or causes of disease, or enhances the therapeutic efficacy of or synergies with another therapeutic agent.

A therapeutically effective dose of a 33-adrenomimetic agent or an agent that contacts and/or interacts with the SMO receptor can depend upon a number of factors known to those of ordinary skill in the art. The dose(s) can vary, for example, depending upon the identity, size, and condition of the subject or sample being treated, further depending upon the route by which the composition is to be administered, if applicable, and the effect which the practitioner desires. It is understood that a medical professional will typically determine the dosage regimen in accordance with a variety of factors. These factors include the cancer and/or tumor from which the subject suffers, the degree of metastasis, as well as the age, weight, sex, diet, and medical condition of the subject.

In some embodiments, the therapeutically effective amount is at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight.

Described herein are methods of treating a subject afflicted with a disease comprising administering to a subject a therapeutically effective amount of a 3-adrenomimetic agent or an agent that contacts and/or interacts with the SMO receptor. The terms “individual”, “patient” and “subject” can be used interchangeably. They refer to a mammal (e.g., a human) which is the object of treatment, or observation. Typical subjects to which a 3-adrenomimemtic agent can be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

The term “administration” can refer to introducing a 3-adrenomimetic agent or an agent that contacts and/or interacts with the SMO receptor into a subject. In general, any route of administration can be utilized. Non-limiting examples of routes of administration comprise parenteral (e.g., intravenous), intraperitoneal, oral, topical, subcutaneous, peritoneal, intraarterial, inhalation, vaginal, rectal, nasal, introduction into the cerebrospinal fluid, or instillation into body compartments. In some embodiments, administration is intraperitoneal. Additionally or alternatively, in some embodiments, administration is parenteral. In some embodiments, administration is intravenous. In other embodiments, administration is orally.

A 3-adrenomimetic agent or an agent that contacts and/or interacts with the SMO receptor or a composition comprising the same can be administered to a subject in need thereof one time (e.g., as a single injection or deposition). Alternatively, administration can be once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12 times per year, or a combination thereof. It can also be administered once or twice daily to a subject for a period of years or until the death of the subject.

A 3-adrenomimetic agent or an agent that contacts and/or interacts with the SMO receptor can be incorporated into a delivery system for administration to a subject. The term “delivery system” can refer to any form of a composition, such as a solid, semi-solid, or liquid, having 3-adrenomimetic agent or an agent that contacts and/or interacts with the SMO receptor or such composition incorporated therein which can deliver 3-adrenomimetic agent or an agent that contacts and/or interacts with the SMO receptor to/into a cell, such as a cancer cell. The delivery system can be a biodegradable delivery system. The composition can be designed to have a desired release rate of a 3-adrenomimetic agent or an agent that contacts and/or interacts with the SMO receptor incorporated therein. The delivery system comprising a 3-adrenomimetic agent or an agent that contacts and/or interacts with the SMO receptor can be administered to a subject as described herein.

The terms “treat,” “treating” or “treatment” can refer to the lessening of severity of a tumor or cancer, delay in onset of a tumor or cancer, slowing the growth of a tumor or cancer, slowing metastasis of cells of a tumor or cancer, shortening of duration of a tumor or cancer, arresting the development of a tumor or cancer, causing regression of a tumor or cancer, relieving a condition caused by a tumor or cancer, or stopping symptoms which result from a tumor or cancer. For example, the invention is directed towards methods of reducing cell viability and/or promoting apoptosis of pancreatic cancer cells or gastric cancer cells by administering to a subject in need thereof a therapeutically effective amount of a 3-adrenomimetic agent or an agent that contacts and/or interacts with the SMO receptor, such as mirabegron.

The approach as described herein (i.e., administration of mirabegron to a subject in need thereof) will provide clinical benefit, defined broadly as any of the following: inhibiting an increase in cell volume, slowing or inhibiting worsening or progression of cancer cell proliferation, reducing primary tumor size, reducing occurrence or size of metastasis, reducing or stopping tumor growth, inhibiting tumor cell division, killing a tumor cell, sensitizing a tumor cell to a drug, radiation, or chemical, inducing apoptosis in a tumor cell, reducing or eliminating tumor recurrence.

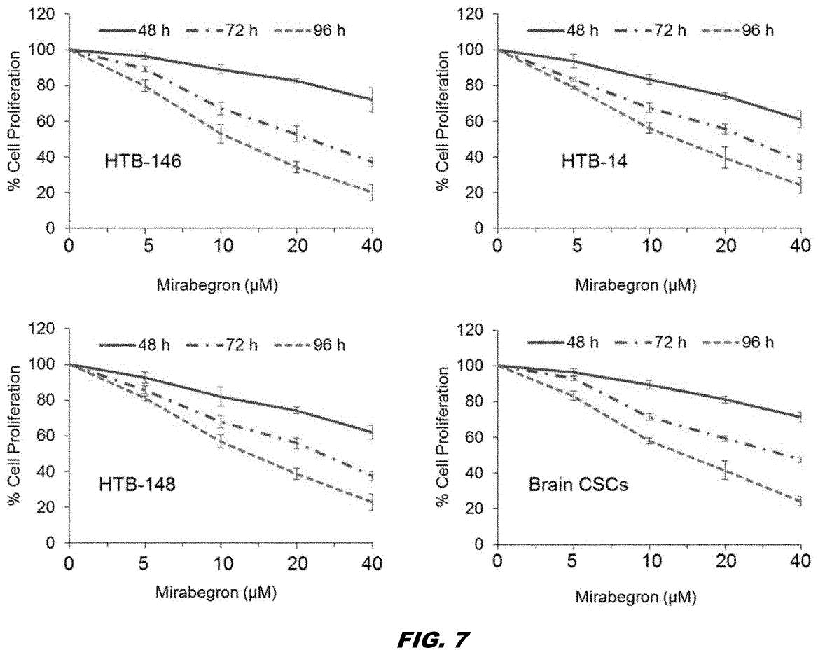

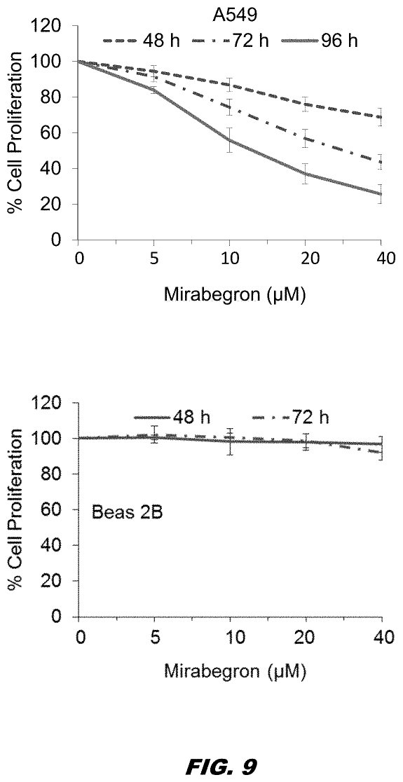

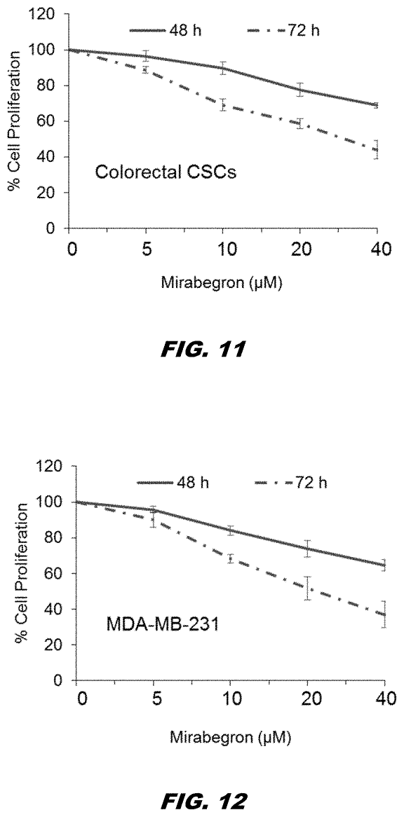

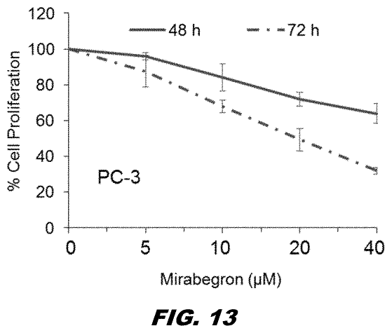

Referring to FIG. 7, for example, Mirabegron inhibits cell proliferation of glioblastoma cell lines and brain CSCs. Further, referring to FIG. 9, for example, Mirabegron inhibits cell proliferation of lung cancer cells, but has no effect on human normal lung epithelial cells. Still further, referring to FIG. 11, FIG. 12, and FIG. 13, for example, Mirabegron inhibits cell proliferation of colotectal CSCs, breast cancer cells, and prostate cancer cells, respectively.

Aspects of the invention are directed towards methods of reducing cell viability of a cancer cell. The term “cell viability” can refer to a measure of the amount of cells that are living or dead, based on a total cell sample. In embodiments, the call can be a brain cancer cell, breast cancer cell, prostate cancer cell, lung cancer cell, leukemia cell, lymphoma cell, ovarian cancer cell, neuroendocrine cancer cell, colon cancer cell, colorectal cancer cell, bladder cancer cell, endometrial cancer cell, neuroblastoma cell, kidney cancer cell, multiple myeloma cell, pancreatic cancer cell, thyroid cancer cell, liver cancer cell, gastric cancer cell, or melanoma cell.

Aspects of the invention are further directed towards methods of preventing the progression of cancer in a subject comprising administering to a subject in need thereof a therapeutically effective amount of a 3-adrenomimetic agent or an agent that contacts and/or interacts with the SMO receptor.

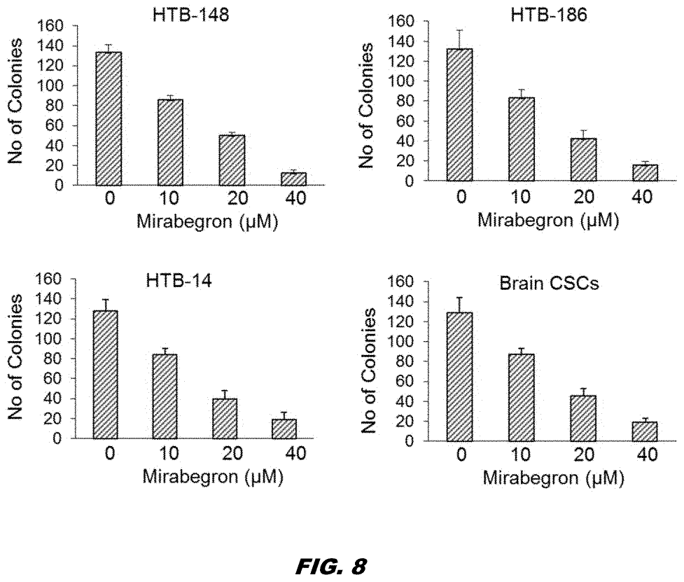

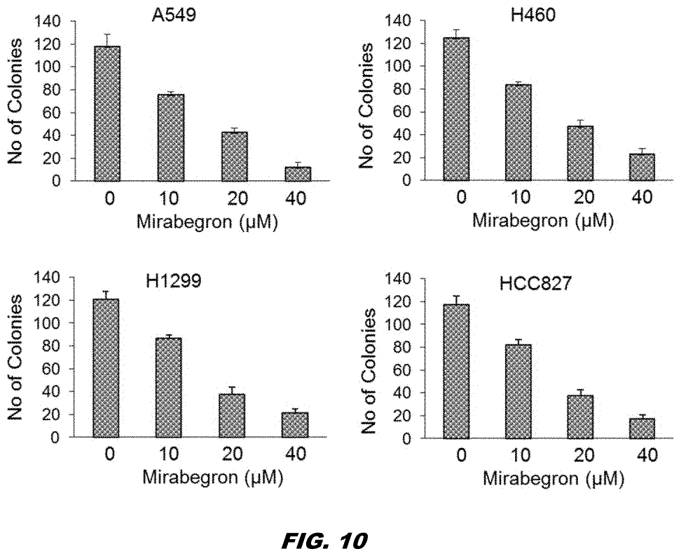

Referring to FIG. 8 and FIG. 10, for example, Mirabegron inhibits colony formation by glioblastoma cells and brain CSCs, and lung cancer cells, respectively.

Embodiments of the invention are directed towards methods of promoting, initiating, and/or enhancing apoptosis of a cancer cell, such as a brain cancer cell, breast cancer cell, prostate cancer cell, lung cancer cell, leukemia cell, lymphoma cell, ovarian cancer cell, neuroendocrine cancer cell, colon cancer cell, colorectal cancer cell, bladder cancer cell, endometrial cancer cell, neuroblastoma cell, kidney cancer cell, multiple myeloma cell, pancreatic cancer cell, thyroid cancer cell, liver cancer cell, gastric cancer cell, or melanoma cell. The term “apoptosis” can refer to a regulated network of biochemical events which lead to a selective form of cell suicide, and is characterized by readily observable morphological and biochemical phenomena, such as the fragmentation of the deoxyribo-nucleic acid (DNA), condensation of the chromatin, which may or may not be associated with endonuclease activity, chromosome migration, margination in cell nuclei, the formation of apoptotic bodies, mitochondrial swelling, widening of the mitochondrial cristae, opening of the mitochondrial permeability transition pores and/or dissipation of the mitochondrial proton gradient.

The invention is also directed towards a method of treating a subject afflicted with a disease, comprising administering to a subject in need thereof a therapeutically effective amount of an agent that contacts and/or interacts with the SMO receptor, wherein the disease is not overactive bladder. Non-limiting examples of such diseases comprise metabolic syndrome, obesity, diabetes, and cancer. Non-limiting examples of such contacts and/or interactions comprise a hydrogen bond, a hydrophobic interaction, an ionic interaction, water bridge formation, or a combination thereof.

Aspects of the invention are directed towards compositions and methods for the treatment of neurological disorders. “Neurological disorder” or “neurological disease” can refer to a general term for diseases of the brain, spine and the nerves that connect them. Non-limiting examples of diseases of the nervous system comprise brain tumors, epilepsy, Parkinson's disease, stroke, and frontotemporal dementia.

Thus, compounds of the invention can be used in the treatment of a neurological disorder, and when used alone or in combination with other compounds, can alleviate, reduce, ameliorate, prevent, or place or maintain in a state of remission of clinical symptoms or diagnostic markers associated with the neurological disorder, and can be used in methods, combinations and compositions provided herein.

A 3-adrenomimetic agent or an agent that contacts and/or interacts with the SMO receptor can also be provided in a kit for treating a disease. In one embodiment, the kit includes (a) a container that contains a 3-adrenomimetic agent, and optionally (b) informational material for treating pancreatic cancer or gastric cancer. The informational material can be descriptive, instructional, marketing or other material that relates to the methods described herein and/or the use of the compound for therapeutic benefit. In an embodiment, the kit includes also includes a second agent for treating a disease. For example, the kit includes a first container that contains a 3-adrenomimetic agent, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the compound, e.g., in a suitable dose, dosage form, or mode of administration (e.g., a dose, dosage form, or mode of administration described herein), to treat a subject who has a disease. The information can be provided in a variety of formats, include printed text, computer readable material, video recording, or audio recording, or any information that provides a link or address to substantive material.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Mirabegron for Cancers and Metabolic Syndrome

Overactive bladder (OAB) symptoms, such as urgency and urgency incontinence, can negatively impact health-related quality-of-life, increase depression and anxiety, and increase healthcare usage. Mirabegron is the only 3-adrenomimetic agent for the treatment of urinary disorders (Overactive bladder) which has been approved for use in Europe and North America. OAB patients receiving mirabegron in a real-world setting reported meaningful improvements in quality of life and health status. No unexpected safety issues were observed, and adverse events were consistent with the known safety profile of mirabegron.

Metabolic syndrome (MetS) comprises a cluster of risk factors that includes central obesity, dyslipidemia, impaired glucose homeostasis and hypertension. Individuals with MetS have elevated risk of type 2 diabetes and cardiovascular disease; thus placing significant burdens on social and healthcare systems. Lifestyle interventions (comprised of diet, exercise, or a combination of both) are routinely recommended as the first line of treatment for MetS. Only a proportion of people respond, and it has been assumed that psychological and social aspects primarily account for these differences. However, the etiology of MetS is multifactorial and stems, in part, on a person's genetic make-up.

Obesity is strongly associated with the development of insulin resistance, which in turn plays a key role in the pathogenesis of obesity-associated cardiometabolic complications, including metabolic syndrome components, type 2 diabetes, and cardiovascular disease. Insulin sensitive tissues, including adipose tissue, skeletal muscle, and liver, are profoundly affected by obesity both at biomolecular and functional levels. Altered adipose organ function may play a fundamental pathogenetic role once fat accumulation has ensued. Modulation of insulin sensitivity appears to be at least in part, related to changes in redox balance and oxidative stress as well as inflammation, with a relevant underlying role for mitochondrial dysfunction that may exacerbate these alterations. Nutrients and substrates as well as systems involved in host-nutrient interactions, including gut microbiota, have been also identified as modulators of metabolic pathways controlling insulin action.

There are no studies demonstrating the clinical uses of Mirabegron in cancer, diabetes, obesity and other metabolic syndrome. Metabolic disorders and obesity have been linked to cancer, and there are no drugs which can be used in both metabolic disorders and cancer.

There is an unmet need to discover new drugs for the treatment of cancer, diabetes, obesity, and metabolic syndrome. Without wishing to be bound by theory, Mirabegron will be very effective in treating cancer, diabetes, obesity, and metabolic syndrome, for example reducing the obesity, diabetes and cancer.

We have demonstrated that Mirabegron kills cancer cells and cancer stem cells. It can affect those signaling pathways which regulate reduce obesity and diabetes. Since it has been approved for overactive bladder, aspects of the invention pertain to a new use for the drug for treating cancer, diabetes, obesity, and other metabolic syndrome.

### Example 2

Mirabegron as a Potent Inhibitor of Hedgehog Signaling Pathways for the Treatment of Cancer

Brain tumor is abnormal growth of cells in the brain. The World Health Organization report estimated 7.6 million people died from cancer accounting for the 13% of all deaths worldwide due to this disease. Glioma is the most lethal adult brain tumor that comprises about 30 percent of all brain tumor and 80 percent of all malignant brain tumors. Although progress has been made to treat the disease but challenges to develop an effective drug devoid of toxicity and side effect still exists in the field of new drug discovery for the treatment of cancer, such as brain cancer, breast cancer, prostate cancer, lung cancer, leukemia, lymphoma, ovarian cancer, neuroendocrine cancer, colon cancer, colorectal cancer, bladder cancer, endometrial cancer, neuroblastoma, kidney cancer, multiple myeloma, pancreatic cancer, thyroid cancer, liver cancer, gastric cancer, or melanoma.

The Hedgehog (Hh) signaling pathway is closely associated with embryonic development, including the formation and maintenance of glioma. Without wishing to be bound by theory, Hh signaling is an important target for cancer therapy since gliomas are important biological factors responsible for cancer invasion, metastasis, drug resistance, and relapse. There are four key components involved in Hh signaling including Hedgehog ligand, Hedgehog Protein patched homolog 1 (PTCH) receptor, smoothened (SMO) cell surface signal transducer, and the Gli transcription factors (downstream effectors). PTCH normally suppresses the activity of SMO, but when hedgehog ligand binds to PTCH, it relieves SMO, which ultimately leads to the transcription of Gli target genes, such as CMYC and BMI-1, along with GLI1 itself. It was reported that up to one third of all human Glioma patients exhibit hyperactive Hh signaling, often due to mutations in the Smo, Ptch1 or Gli target genes. Several antagonists targeting SMO are in advanced-stage clinical trials that inhibit tumor growth as well as symptomatic relief to patients. Thus SMO receptor has emerged as an attractive therapeutic target that can effectively generate the inhibitor for the development of anti-tumor drug. Recently, molecular docking studies have been exploited to identify the direct binding of inhibitors to the transmembrane helices of SMO.

However, this whole new drug development for cancer is an awful process. The evolution of a whole new drug is a very uncertain, slow, and costly affair with high unsuccessful rate, and has to undergo a difficult approval procedure by the federal agencies. Identifying new uses of drugs established for the treatment of other, non-related disorder/disease is one of the novel approach of therapeutics. There are the range of drugs like Sildenafil, Plerixafor, and Rapamycin which were successfully repurposed and benefiting the society for the diseases other than for which they are developed.

In the current study, we first employed Virtual screening (VS) on the transmembrane active pocket of SMO for 4199 drugs (FDA approved drugs and drugs approved outside the United States). Among the screened drugs we further manually screened based on the interaction analysis at the binding pockets of both the enzyme. For instance, in case of SMO, only those drugs were filtered that were shown enough interaction through hydrogen bonds at the catalytic machinery of this enzyme. Interestingly, four drugs (Cynarine, Conivaptan, Silodosin, and Mirabegron) stand out as the top hit. Finally, after this rigorous in silico filter screening protocol Mirabegron was identified as a potential compound that could target the Hedgehog (Hh) signaling pathway by targeting the SMO receptor.

Mirabegron is the only 3-adrenomimetic agent for the treatment of urinary disorders (Overactive bladder) which has been approved for use in Europe and North America. Overactive bladder patients receiving Mirabegron in a real-world setting reported meaningful improvements in quality of life and health status. No unexpected safety issues were observed, and adverse events were consistent with the known safety profile of Mirabegron.

There are no studies demonstrating Mirabegron's clinical uses in cancer. Since it has been approved for overactive bladder, it can be used as a new drug for cancer and another disease. In current work, we have demonstrated that it affect Hedgehog signaling pathways which was described to be closely associated with tumor initiation.

Materials and Methods

Protein Preparation and Grid Generation:

Without wishing to be bound by theory, we search the new hits with a dual binding affinity with SMO receptor. The 3d structure of SMO receptor (4JKV) complexed with 4-fluoro-N-methyl-N-{1-[4-(1-methyl-1H-pyrazole-5-yl)phthalazin-1-yl]piperidin-4-yl}-2-(trifluoromethyl)benzamide [LY2940680]. The retrieved structure consists of water molecules, co-crystallized ligands, metal ions and co-factors. Protein preparation wizard was used for the crystal structure preparation of SMO. The solved structure is a dimer, and only chain A was used for the docking study. The binding site was identified by determining the position of the ligand LY-2940680. The structure was processed by deletion of water molecule beyond 5 Å, filling of missing side chain and loops, assign of bond order and the addition of hydrogen atoms into the crystal structure. Further, structure refinement and restrain minimization were done using OPLS force field. The grid was generated by using the co-crystallized ligand present as the centroid of the active site having a box volume of 10*10*10 Å. Further, virtual screening and docking of prepared ligand were performed using the generated grid, and lowest energy docked conformation was retained.

Ligands for Virtual Screening:

Drug central a database of FDA approved drugs and drugs approved outside United States (www.drugcentral.org) were used as ligands for the virtual screening to identify new potential hits. The database contains 4199 compounds as of October 2017 were downloaded as SD file and further optimized using the big prep module of Schrodinger 2016-1 using default parameter at pH 7.4.

Structure-Based Virtual Screening:

Virtual screening of chemical databases is a widely used approach for the identification of potential lead against the desired target. The prepared ligand was virtual screened using the Glide module of Schrodinger. The virtual screening study was performed in three sequential steps namely high throughput virtual screening (HTVS), standard precision (SP) and extra precision (XP) docking using the filter criteria of 10%, 10%, and 25% respectively in each step.

Result and Discussion:

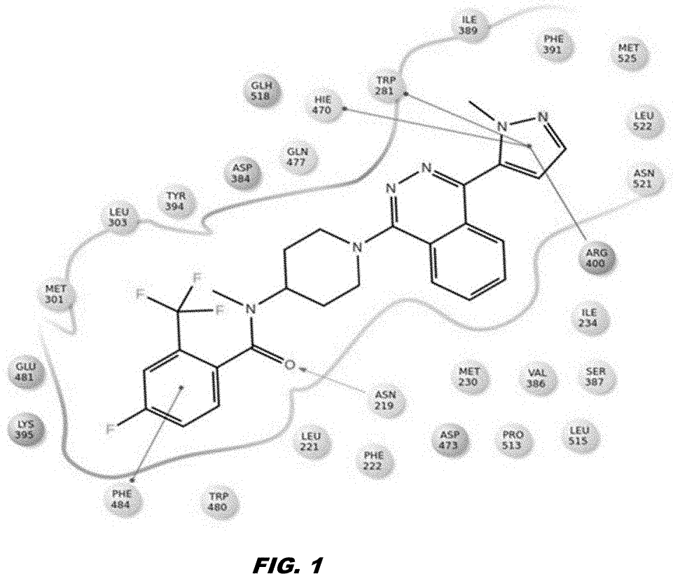

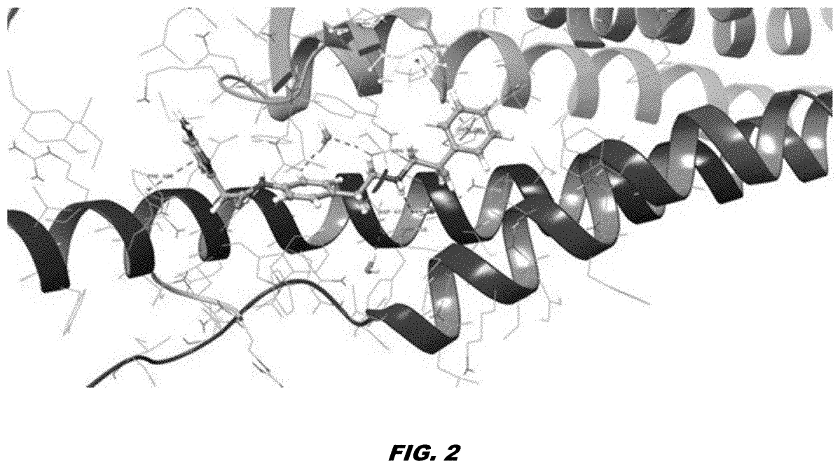

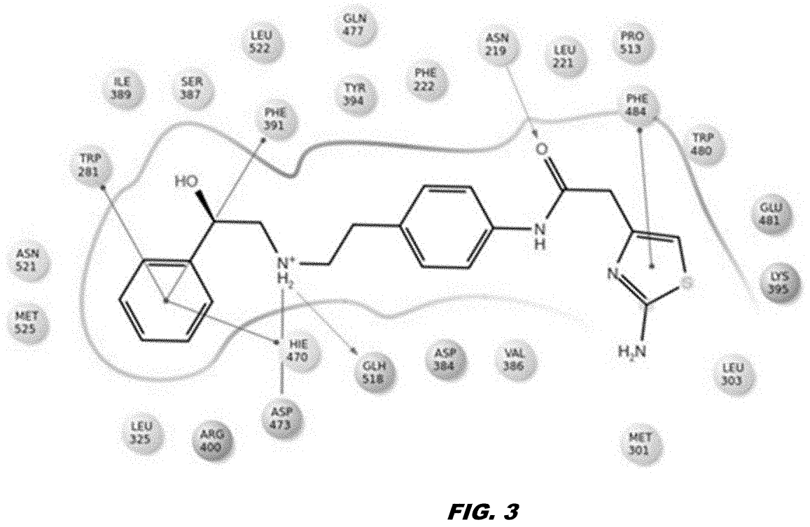

Virtual Screening and Hit Identification:
a) Docking investigation and Binding energy calculation of identified hits:
   Using HTVS among the selected molecules, four hits namely Cynarine, Conivaptan, Silodosin, and Mirabegron were found to show the good binding affinity for SMO receptor. The identified hits were further docked by Glide XP protocol for refinement of the glide score. After docking, we evaluate the mechanistic mode of interaction with the target proteins. 2D and 3D structure of the identified hits are shown in FIG. 1-3. The docking results revealed that LY2940680 has highest XP score of −11.602 Kcal/mole against SMO. On the other hand, Mirabegron shows XP score −9.971 Kcal/mole. The details of the interaction energy and interacting residues with all the identified hits are shown in Table 1.
b) Molecular docking and interaction analysis of Mirabegron and LY2940680 on SMO receptor active transmembrane binding site:
   The SMO receptor binding pocket has a narrow and long shape and is connected to the extracellular aqueous environment through a small opening formed by an extracellular domain (ECD) composed of an extracellular cysteine-rich domain (CRD) and an ECD linker domain. Without wishing to be bound by theory, this orifice facilitates small-molecule ligand entry into the transmembrane core region. Residues from the extracellular tips of helices I, II, V, and VII interact with phthalazine ring of LY2940680 via the hydrogen bond to Arg400. Most of the other contact residues belong to the ECD linker domain and ECLs. The ligand was mediating the 3n interaction with Trp-281, Hie-470, Phe-484, Arg-400, it also showed hydrophobic interaction with other important residues have a role in the conformational properties and dynamics of the pocket.
   Mirabegron showed Hydrogen bonding interaction with Asn219, 1 salt Bridge with Asp-473, 3n interaction with Trp-281, Phe-391, Phe-484, and His-470. Whereas, it also showed hydrophobic interaction with other important residues.

TABLE 1
Docking Analysis of Standard drug (LY2940680) and Mirabegron on PDB: 4JKV.

| | | Glide Score | | |
|---|---|---|---|---|
| Protein | Ligand | (Kcal/mole) | Interacting Residue | Bond Type |
| 4JKV | (LY2940680) | −11.606 | Asn-521, Arg-400, Ile-234, Ser- | 1 Hydrogen bonding, |
| | | | 387, Val-386, Leu-515, Pro- | 3 [symbol] interaction(Trp- |
| | | | 513, Met-230, Asp-473, Phe- | 281, Hie-470, Phe- |
| | | | 222, Leu-221, Trp-480, Phe- | 484, Arg-400, other |
| | | | 484, Lys-395, Glu-481, Met- | hydrophobic interaction |
| | | | 301, Leu-303, Tyr-394, Asp- | |
| | | | 384, Gln-477, Glh-518, His- | |
| | | | 470, Trp-281, Ile-389, Phe- | |
| | | | 391, Met-525, Leu-522 | |
| | Mirabegron | −9.971 | Leu-303, Met-301, Val-386, Asp- | 3 Hydrogen |
| | | | 384, Asp-473, His-470, Arg- | bonding, 1salt Bridge |
| | | | 400, Leu-325, Met-525, Asn- | (Asp-473) 3 [symbol] |
| | | | 521, Trp-281, Ile-389, | interaction (Trp- |
| | | | Ser-387, Phe-391, Leu-522, Tyr- | 281, Phe-391, Phe- |
| | | | 394, Gln-477, Phe-222, Leu- | 484, His-470), other |
| | | | 221, Pro-513, Phe-484, Trp- | hydrophobic interaction |
| | | | 480, Glu-481, Lys-395 | |

Conclusion:

As drug repurposing is an effective strategy to decrease the cost and time required for new drug discovery and development, here we undertook Virtual screening of approved drugs for targeting cancer. A series of in silico techniques like structure-based and ligand-based Virtual screening were employed to identify suitable drugs to act as a potent inhibitor of Hedgehog signaling pathways the treatment for Cancers that work through SMO receptor. Based on these in silico experiments, Mirabegron, an approved drug for overactive bladder, was identified as a suitable candidate for testing in cancer cell line.

### Example 3

Experimental:

The binding stability and binding pattern of the docked complex of Mirabegron were analyzed on human smoothened 7TM receptor (PDB Code: 4JKV) protein. The 10 ns molecular dynamics simulation run was performed from the docked complex of ligand (Mirabegron) on the protein using the Desmond module of Schrödinger Maestro 10.5.014 program. The cubic simulation box was prepared to build the system. The TIP3P explicit water model was used, and the minimum distance of 10 Å was set between box wall and protein-ligand complex. The system was neutralized with the addition of counter ions, and the isosmotic salt environment was provided with 0.15M NaCl. The system was further minimized with maximum 2000 iterations with convergence criteria of 1 kcal/mol/Å. The minimized docked complex was further subjected to molecular dynamics simulations for 10 ns. The recording interval energy was kept at 1.2 ps, and the trajectory was set at 9.6. The simulation run was performed at the constant number of atoms (N), pressure (P) and temperature (T) (NPT) ensemble at the temperature of 300K and 1.013 bars atmospheric pressure. The generated trajectory was utilized to generate simulation interaction diagrams and analyze the results.

Results and Discussion:

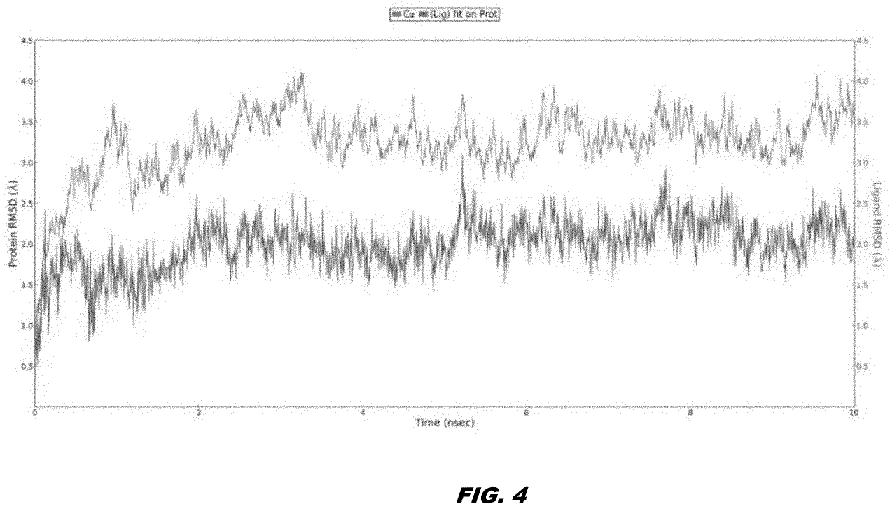

The preceding findings of molecular docking analysis made it worthwhile to confirm the binding mode stability and integrity of complex with molecular dynamics simulations run. The simulation run of 10 ns was performed for Mirabegron independently on human smoothened 7TM receptor (PDB Code: 4JKV) protein. The overall stability of the system was evaluated by RMSD (root mean square deviation) and RMSF (root mean square fluctuation) calculations. RMSD calculations are based on the alignment of protein backbone with the reference initial backbone structure. The RMSD calculations provide insights of structural stability and conformations throughout the simulations run. The results of the RMSD values for Mirabegron simulation runs confirmed the energetically stable trajectories and integrity throughout the simulations with average fluctuations in the range of 1-3 Å (FIG. 4). Further interactions factions and accommodation in the active binding site for all the ligand was analyzed through the simulations run.

Simulation interaction diagram module was utilized to generate stacked bar chart, which showed the normalized interaction over the course of trajectory. The stacked bar chart showed four types of ligand-protein interactions including Hydrogen Bonds, Hydrophobic, Ionic, and Water Bridges. A schematic protein-ligand contacts diagram was also generated with contacts of more than 30% throughout the simulation run. Further, a timeline representation of all amino acid residues with ligand was also generated and analyzed.

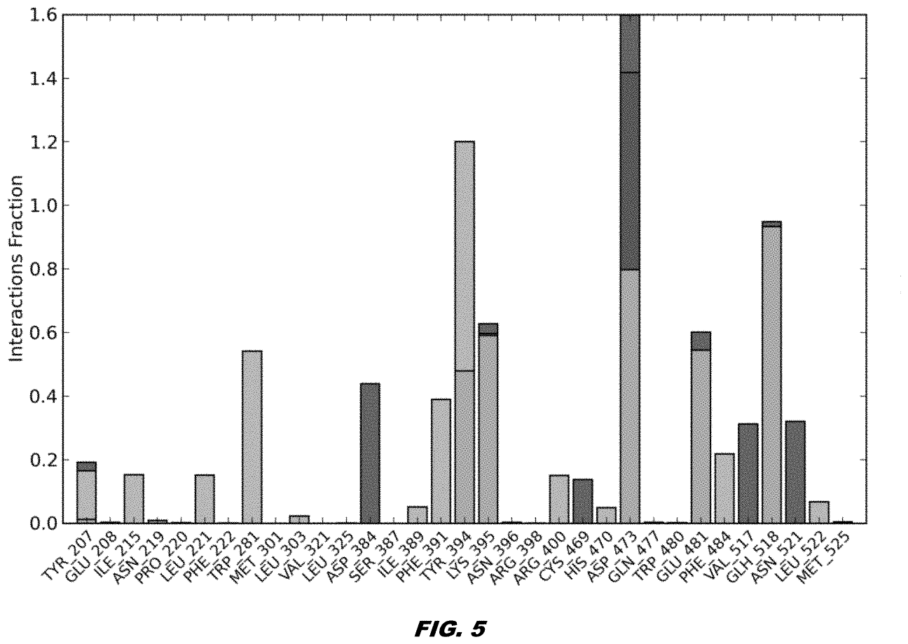

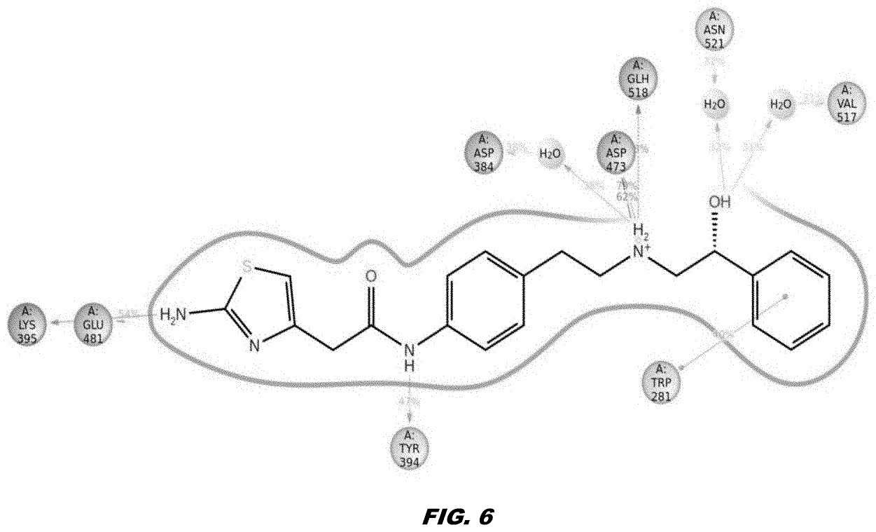

Molecular dynamics studies of Mirabegron on human smoothened 7TM receptor (PDB Code: 4JKV) protein showed the H-bonding interactions with Glh518, Asp473, and Glu481 remained intact and completely stable for the maximum period of simulation run (FIG. 5 and FIG. 6). The free amine group of Mirabegron is making H-bonding contact with Glu481 and Lys395 while the phenyl ring is showing n-n stacking interaction with Trp281. It also displays Hydrophobic interaction with important amino acids Arg400 and Phe484.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of treating a subject afflicted with a disease, comprising administering to a subject in need thereof a therapeutically effective amount of a 3-adrenomimetic agent, wherein the disease is not overactive bladder.

2. A method of preventing the progression of cancer in a subject, comprising administering to a subject in need thereof a therapeutically effective amount of a 3-adrenomimetic agent.

3. The method of claim 2, wherein the agent induces apoptosis of a cancer cell, inhibits proliferation of a cancer cell, inhibits colony formation, inhibits migration of a cancer cell, or a combination thereof.

4. The method of claim 3, wherein the cancer cell comprises a cancer stem cell.

5. The method of claim 1 or claim 2, wherein the 3-adrenomimetic agent comprises mirabegron.

6. The method of claim 1, wherein the disease comprises metabolic syndrome, obesity, diabetes, cancer.

7. The method of claim 2 or claim 6, wherein the cancer comprises a brain cancer, breast cancer, prostate cancer, lung cancer, leukemia, lymphoma, ovarian cancer, neuroendocrine cancer, colon cancer, colorectal cancer, bladder cancer, endometrial cancer, neuroblastoma, kidney cancer, multiple myeloma, pancreatic cancer, thyroid cancer, liver cancer, gastric cancer, or melanoma.

8. The method of claim 7, wherein the brain cancer comprise glioma.

9. The method of claim 1 or claim 2, wherein the agent contacts and/or interacts with the SMO receptor.

10. The method of claim 9, wherein contact and/or interaction comprises a hydrogen bond, a hydrophobic interaction, an ionic interaction, water bridge formation, or a combination thereof.

11. The method of of claim 9, wherein the agent contacts and/or interacts with the SMO receptor at Tyr394, Lys395, Asp473, Glu481, Glh518 or any combination thereof.

12. The method of claim 9, wherein the agent contacts and/or interacts with the SMO receptor at Tyr207, Ile215, Leu221, Trp281, Ile389, Phe391, Trp394, Arg400, His470, Phe484, Leu522, or any combination thereof.

13. The method of claim 1 or claim 2, wherein the agent contacts and/or interacts with the SMO receptor at Asp384, Val517, Asn 521, or any combination thereof.

14. A method of treating a subject afflicted with a disease, comprising administering to a subject in need thereof a therapeutically effective amount of an agent that contacts and/or interacts with the SMO receptor, wherein the disease is not overactive bladder.

15. The method of claim 14, wherein contact and/or interaction comprises a hydrogen bond, a hydrophobic interaction, an ionic interaction, water bridge formation, or a combination thereof.

16. The method of claim 14, wherein the agent contacts and/or interacts with the SMO receptor at Tyr394, Lys395, Asp473, Glu481, Glh518, Tyr207, Ile215, Leu221, Trp281, Ile389, Phe391, Trp394, Arg400, His470, Phe484, Leu522, Asp384, Val517, Asn 521 or any combination thereof.

17. The method of claim 14, wherein treating comprises preventing the progression of cancer in the subject.

18. The method of claim 14, wherein the agent induces apoptosis of a cancer cell, inhibits proliferation of a cancer cell, inhibits colony formation, inhibits migration of a cancer cell, or a combination thereof.

19. The method of claim 14, wherein the agent comprises a 3-adrenomimetic agent.

20. The method of claim 14, wherein the agent comprises mirabegron.

21. The method of claim 14, wherein the disease comprises metabolic syndrome, obesity, diabetes, cancer.

22. The method of claim 21, wherein the cancer comprises a brain cancer, breast cancer, prostate cancer, lung cancer, leukemia, lymphoma, ovarian cancer, neuroendocrine cancer, colon cancer, colorectal cancer, bladder cancer, endometrial cancer, neuroblastoma, kidney cancer, multiple myeloma, pancreatic cancer, thyroid cancer, liver cancer, gastric cancer, or melanoma.

23. The method of claim 22, wherein the brain cancer comprise glioma.

24. A method of treating a neurological disorder, the method comprising administering to a subject afflicted with a neurological disorder a therapeutically effective amount of a compound described herein.
